(19) United States
(12) Patent Application Publication
(10) Pub. No.: US 2016/0145178 A1
(43) Pub. Date: May 26, 2016

(54) METHODS AND APPARATUSES FOR GENERATING A POLYOL FROM WHOLE BIOMASS

(71) Applicant: UOP LLC, Des Plaines, IL (US)

(72) Inventors: Tom N Kalnes, LaGrange, IL (US); John Qianjun Chen, Glenview, IL (US); Joseph Anthony Kocal, Glenview, IL (US)

(21) Appl. No.: 14/549,209

(22) Filed: Nov. 20, 2014

(51) Int. Cl. C07C 27/04; B01J 8/24; B01J 8/02

(52) U.S. Cl. CPC C07C 27/04; B01J 8/02; B01J 8/24; B01J 2208/02

(57) ABSTRACT

Methods and apparatuses for catalytically generating a polyol from whole biomass are provided. An exemplary method includes the steps of: depolymerizing at least a portion of lignin present in a stream that includes whole biomass; generating an effluent that includes depolymerized lignin and a saccharide; separating the effluent to generate a depolymerized lignin stream and a saccharide process stream, wherein the saccharide process stream includes a saccharide and an amount of lignin that is reduced relative to an amount of lignin present the effluent; and contacting the saccharide process stream with a saccharide-to-polyol catalyst system under conditions suitable for the catalytic conversion of saccharide to polyol.

### TECHNICAL FIELD

The technical field generally relates to methods and apparatuses for generating a polyol from whole biomass, and more particularly to methods and apparatuses for generating a polyol from whole biomass with a catalyst system.

### BACKGROUND

Polyols are valuable materials with uses such as preparation of purified terephthalic acid (PTA)/polyethylene terephthalate polymers (PET), cold weather fluids, cosmetics and many others. Generating polyols from cellulose instead of olefins can be a more environmentally friendly and economically attractive process. Previously, polyols have been generated from polyhydroxy compounds. Polyols have also been catalytically generated directly from cellulose in batch type and continuous processes. Catalytic conversion of cellulose into ethylene glycol over supported carbide catalysts, such as tungsten carbide catalysts also has been achieved. However, certain components in whole biomass, such as lignin or metal impurities, have a negative impact on catalyst performance and lifespan.

Accordingly, it is desirable to provide methods and apparatuses for catalytic polyol production from whole biomass with reduced contact of lignin and metal impurities with the catalyst. Furthermore, other desirable features and characteristics of the present invention will become apparent from the subsequent detailed description of the invention and appended claims, taken in conjunction with the accompanying drawings and this background of the invention.

### BRIEF SUMMARY

Methods and apparatuses for catalytically generating a polyol from whole biomass are provided. An exemplary method includes the steps of: depolymerizing at least a portion of lignin present in a stream that includes whole biomass; generating an effluent that includes depolymerized lignin and a saccharide; separating the effluent to generate a depolymerized lignin stream and a saccharide process stream, wherein the saccharide process stream includes a saccharide and an amount of lignin that is reduced relative to an amount of lignin present the effluent; and contacting the saccharide process stream with a saccharide-to-polyol catalyst system under conditions suitable for the catalytic conversion of saccharide to polyol.

In another embodiment, an exemplary method includes the steps of: depolymerizing at least a portion of lignin present in a stream that includes whole biomass; generating a first effluent that includes depolymerized lignin and a saccharide; separating the effluent to generate a depolymerized lignin stream and a saccharide process stream, wherein the saccharide process stream includes a saccharide and an amount of lignin that is reduced relative to an amount of lignin present the effluent; contacting the saccharide process stream with a particulate saccharide-to-polyol catalyst system in a slurry reactor under conditions suitable for the catalytic conversion of saccharide to polyol; generating a second effluent comprising a polyol and saccharide-to-polyol catalyst system particles; separating the saccharide-to-polyol catalyst system particles from the second effluent; and recycling the separated saccharide-to-polyol catalyst particles back to the slurry reactor.

In another embodiment, an exemplary apparatus for the catalytic generation of a polyol from whole biomass comprises: a first reaction zone configured to receive an input steam comprising whole biomass, contact the input stream with conditions suitable to depolymerize lignin present in the input stream, and generate a first effluent comprising depolymerized lignin and a saccharide; a first separation zone in fluid communication with the first reaction zone, the first separation zone configured to receive the first effluent and separate the first effluent into a saccharide process stream and a depolymerized lignin stream, wherein the saccharide process stream has an amount of lignin that is reduced relative to an amount of lignin present in the first effluent; and a second reaction zone in fluid communication with the first separation zone; the second reaction zone configured to receive the saccharide process stream, contact the saccharide process stream with a saccharide-to-polyol catalyst under conditions suitable to generate a polyol, and generate a second reaction zone effluent including a polyol.

### BRIEF DESCRIPTION OF THE DRAWING

The various embodiments will hereinafter be described in conjunction with the following drawing FIGURE, wherein:

FIG. 1 is a block diagram of an apparatus used for catalytic polyol generation from whole biomass in accordance with exemplary embodiments described herein. Various optional features, discussed below, are shown.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

Various embodiments provided herein relate to methods and apparatuses for the catalytic generation of at least one polyol from a feedstock comprising a whole biomass, and in some embodiments, whole cellulostic biomass. As used herein, the term “cellulostic biomass” refers to biological material that contains cellulose. Cellulostic biomass is typically derived from plant matter and contains three major polysaccharides, cellulose, pectin, and hemicellulose. Polymeric lignin is also typically present in significant amounts. Further, impurities, such as metals and ash, are typically present as well. Cellulostic biomass used in methods and apparatuses described herein may be whole biomass; that is, cellulostic biomass used in methods and apparatuses described herein may containing cellulose, hemicellulose, and polymeric lignin.

Conventional methods and apparatuses for the catalytic generation of polyols from whole cellulostic biomass rely on reactions that occur between a saccharide, water and hydrogen. As used herein, the term “saccharide” is meant to include any of monosaccharides, disaccharides, oligosaccharides, and polysaccharides. A saccharide may be edible, inedible, amorphous, or crystalline in nature. Polysaccharides may consist of one or more monosaccharides linked by glycosidic bonds. Examples of polysaccharides include glycogen, cellulose, hemicellulose, starch, chitin, and combinations thereof.

Conducting catalytic generation of polyols from saccharides in the presence of lignin and various impurities typically found in whole biomass (e.g., various metals and ash) is detrimental to catalyst performance and lifespan. Without wishing to be bound by theory, it is believed that this detrimental effect is the result of lignin, ash, and metal impurities filling pores in the catalyst material and thus blocking access to catalytically active sites. Additionally, it is believed that alkali and alkaline earth cations may exchange with acidic sites thereby reducing activity. To reduce these detrimental effects on the catalyst, methods and apparatuses described herein utilize at least a first reaction zone, a first separation zone, and a second reaction zone. The first reaction zone receives an input stream comprising whole cellulostic biomass and treats the input stream to depolymerize lignin contained therein. An effluent from the first reaction zone thus contains a saccharide and depolymerized lignin. This effluent is directed to the first separation zone to separate depolymerized lignin (and in some embodiments, additionally to separate various impurities, such as metals and ash) from the effluent, generating a saccharide process stream, a depolymerized lignin stream, and optionally a waste stream. The saccharide process stream comprises a saccharide and an amount of lignin that is reduced relative to the amount of lignin in the effluent. In embodiments where a waste stream comprising ash and metal impurities is also generated, the saccharide process stream comprises amounts of ash and metal impurities that are also reduced relative to amounts of ash and metal impurities present in the effluent. The saccharide process stream is then directed to a second reaction zone, where saccharides are catalytically converted into one or more polyols.

One challenge in processing whole cellulostic biomass to generate polyols is that whole cellulostic biomass is typically in a solid state. Therefore, pretreatment of the whole cellulostic biomass may be performed in order to facilitate continuous transporting. In some embodiments, pretreatment operations include sizing, drying, grinding, and combinations thereof. Whole cellulostic biomass may be processed by one or more pretreatment techniques into solid particles of a size that may be passed or moved through a continuous process using a liquid or gas flow, or mechanical processing as part of the input stream into a first reaction zone.

As indicated above the whole cellulostic biomass-containing input stream is introduced into a first reaction zone where lignin is depolymerized. It is intended that the first reaction zone is not limited to utilizing a specific lignin depolymerization technique. Rather, any conventional lignin depolymerization technique may be utilized, so long as depolymerization conditions do not destroy all saccharides in the input stream. In some embodiments, suitable lignin depolymerization techniques include hot water treatment, steam treatment, hydrolysis, pyrolysis, thermal treatment, chemical treatment, biological treatment, catalytic treatment, pressure treatment, and combinations thereof. In some embodiments, chemical treatment comprises acid catalyzed hydrolysis or base catalyzed hydrolysis. One particular example of a chemical treatment is mild acid hydrolysis. In some embodiments utilizing acidic hydrolysis, the hydrolysis solution may be continuously or periodically replentished with fresh acid to replace acid that is neutralized by alkali or alkaline earth cations. In some embodiments, catalytic treatments include catalytic hydrolysis, catalytic hydrogenation, or both. In some embodiments, biological treatment includes enzymatic hydrolysis. In some embodiments, pressure treatment comprises treating a feedstock with a protocol that includes a pressurization and/or depressurization step. For instance, in some embodiments, a pressure treatment comprises steam explosion, ammonia fiber explosion, hot hydrogen explosion, or the like. In some embodiments, a pressure treatment is used together with a thermal or chemical treatment to achieve a desired degree of lignin depolymerization.

In some embodiments, the first reaction zone comprises a depolymerization reactor that utilizes a conventional acidic or bifunctional catalyst. As used herein, a bifunctional catalyst is a catalyst that allows for simultaneous reduction in molecular weight (i.e., depolymerization) and addition of hydrogen to polymeric lignin. In some embodiments, an appropriate solvent, such as an aromatic hydrocarbon, may be added to the reactor system to enhance depolymerization reaction(s). In some embodiments, the depolymerization reactor is a slurry reactor.

Lignin is depolymerized in the first reaction zone, and a first effluent containing depolymerized lignin, saccharides (including e.g., polysaccharides), and metals and ash impurities is generated. The first effluent is directed to the first separation zone, where the first effluent is separated into at least two streams: a saccharide process stream and a depolymerized lignin stream. In some embodiments, the first separation zone also generates a waste stream comprising ash and/or metal impurities from the whole cellulostic biomass. The first separation zone may utilize any suitable separation technique. For instance, in some embodiments, the first separation zone may employ hydrogen-stripping, steam stripping, and/or liquid extraction of the depolymerized lignin using a polar solvent. In some specific embodiments, the polar solvent may be an ionic liquid.

The saccharide process stream resulting from this separation comprises one or more saccharides and is directed to the second reaction zone. The second reaction zone is configured to receive the saccharide process stream and expose a reaction mixture comprising one or more saccharides, water, and hydrogen to conditions suitable for the catalytic conversion of saccharide to polyol. However, saccharides, including cellulose, are thermally sensitive. That is, exposing saccharides to excessive heating may result in undesired thermal reactions, e.g., charring. Thus, in some embodiments, various apparatus configurations are selected to avoid thermal damage to saccharides in the saccharide process stream. For instance, in some embodiments the saccharide process stream and a hydrogen stream are provided separately to the second reaction zone. In some embodiments, a portion of hydrogen may also be added to the saccharide process stream. Further, water may be present in the saccharide process stream, the hydrogen stream, or both. Additionally, water may be added to the second reaction zone via an optional independent water stream.

In these embodiments, providing the saccharide process stream and at least a portion of the hydrogen to the second reaction zone as two independent streams allows for control of the temperature of the second reaction zone without subjecting saccharides in the saccharide process stream to excessive heating. Specifically, the independent hydrogen stream may be heated to a temperature in excess of the reaction temperature of the second reaction zone such that when the independent hydrogen stream and saccharide process stream are introduced into the second reaction zone and mixed, the resulting reaction mixture is at or above the necessary reaction temperature for saccharide to polyol conversion. Thus, the temperature of the saccharide process stream may be controlled so as not to exceed the decomposition temperature of cellulose or so as not to exceed the charring temperature of cellulose. Further, the saccharide process stream, the hydrogen stream, or both may be pressurized to reaction pressure before being introduced into the second reaction zone.

In some embodiments, the saccharide to polyol catalytic conversion is conducted with a conventional saccharide-to-polyol catalyst system. Conventional catalyst systems typically comprise at least two active metal components: a first active metal component of elemental platinum (Pt), elemental palladium (Pd), elemental ruthenium (Ru), or a combination thereof; and a second active metal component of molybdenum (Mo), tungsten (W), vanadium (V), nickel (Ni), cobalt (Co), iron (Fe), tantalum (Ta), niobium (Nb), titanium (Ti), chromium (Cr), zirconium (Zr), or any combination thereof, wherein the second active metal component is in the elemental state, a carbide compound, a nitride compound, a phosphide compound, or any combination thereof. The saccharide-to-polyol catalyst system may further comprise a support material which can be a powder, or formed in specific shapes such as spheres, extrudates, pills, pellets, tablets, irregularly shaped particles, monolithic structures, catalytically coated tubes, or catalytically coated heat exchanger surfaces. Examples of suitable support materials include the refractory inorganic oxides including but not limited to silica, alumina, silica-alumina, titania, zirconia, magnesia, clays, zeolites, molecular sieves, etc. As will be understood, silica-alumina is not a mixture of silica and alumina but rather is an acidic and amorphous material that has been cogelled or coprecipitated. Carbon and activated carbon may also be employed as support materials. Specific suitable supports include carbon, Al₂O₃, ZrO₂, SiO₂, MgO, CeₓZrO_{y}, TiO₂, SiC. Mixtures of any of these or any other suitable support materials may also be used.

Alternatively or in addition, compound catalyst systems such as those described in U.S. Pat. No. 8,222,462 and U.S. Pat. No. 8,222,463 may be used for saccharide to polyol catalytic conversion. For instance, a compound catalyst system comprising an unsupported component comprising a compound selected from the group consisting of a tungsten compound, a molybdenum compound, and any combination thereof, and a supported component comprising an active metal component selected from the group consisting of Pt, Pd, Ru, Rh, Ni, Ir, and combinations thereof on a solid catalyst support. Suitable solid catalyst supports include those provided above.

In some embodiments, the saccharide-to-polyol catalyst system may reside within the second reaction zone. In other embodiments, the saccharide-to-polyol catalyst system may continuously or intermittently pass through the second reaction zone. In some embodiments, the second reaction zone includes a conventional reactor system. Exemplary suitable reactor systems include ebullating catalyst bed systems, immobilized catalyst reaction systems having catalyst channels, augured reaction systems, fluidized bed reactor systems, mechanically mixed reaction systems, or slurry reactor systems, also known as three phase bubble column reactor systems. In some specific embodiments, the second reaction zone employs a slurry reactor system. In such embodiments, the saccharide-to-polyol catalyst system may be mixed with the saccharide process stream and a sufficient amount of water to form a slurry prior to introduction into the slurry reactor. Catalytic conversion occurs within the slurry reactor and the catalyst system is transported out of the reactor as a component in an effluent stream. The slurry reactor system may be operated at temperatures from about 100° C. to about 350° C. and the hydrogen pressure may be greater than about 150 psig. In some embodiments, the temperature in the slurry reactor system may range from about 150° C. to about 350° C., such as about 200° C. to about 280° C. In some embodiments, the slurry reactor is operated with a water to saccharide weight ratio of about 1 to about 100, such as about 1 to about 20, such as about 1 to about 5. In some embodiments, the slurry reactor is operated with a catalyst to saccharide weight ratio of greater than about 0.005, such as greater than about 0.01, such as greater than about 0.1. In some embodiments, the slurry reactor is operated with a pH of less than about 10. In some embodiments, the slurry reactor is operated with a residence time of greater than 5 minutes. In some embodiments, the slurry reactor is operated such that any combination of these operating conditions are met.

In a specific exemplary embodiment, the slurry reactor is operated with a water to saccharide weight ratio of about 1 to about 20 and with a catalyst to saccharide weight ratio of greater than about 0.01. In yet another specific exemplary embodiment, the slurry reactor is operated with a water to saccharide weight ratio of about 1 to about 5 and with a catalyst to saccharide weight ratio of greater than about 0.1.

Furthermore, the materials which make up at least a portion of the second reaction zone are selected to be compatible with the reactants and the desired products within the range of operating conditions. Examples of suitable metallurgy for the second reaction zone include titanium, zirconium, stainless steel, carbon steel having hydrogen embrittlement resistant coating, and carbon steel having corrosion resistant coating. In one embodiment, at least a portion of the second reaction zone includes zirconium clad carbon steel.

Within the second reaction zone and at operating conditions, the reactants (i.e., saccharide, hydrogen, and water) proceed through catalytic conversion reactions to produce at least one polyol. In some embodiments, the product polyol includes ethylene glycol, propylene glycol, or a combination thereof. At least one co-product may also be produced. Such co-products may be alcohols, organic acids, aldehydes, monosaccharides, polysaccharides, phenolic compounds, hydrocarbons, glycerol, depolymerized lignin, carbohydrates, and proteins. In some embodiments, a plurality of co-products may be produced. Some of the co-products may have commercial value and may be isolated and recovered in addition to the product polyols. Co-products may also be reaction intermediates which, in some embodiments, are separated from the second reaction zone effluent and recycled back to the second reaction zone.

Unreacted hydrogen, water, and saccharides may also be present in the second reaction zone effluent. In some embodiments, unreacted hydrogen, water, saccharides, or any combination thereof, are separated and recycled. In some embodiments, hydrogen is separated from the effluent stream before water is separated from the effluent stream. The separated hydrogen may be recycled to one or more of a number of different locations within the process depending upon the specific embodiment employed. For example, the separated hydrogen maybe recycled to a reactor in the second reaction zone. The recycled hydrogen may be combined with fresh hydrogen or make-up hydrogen before being introduced into a reactor in the second reaction zone, or recycled hydrogen may be introduced to a reactor in the second reaction zone independently of fresh hydrogen or make-up hydrogen. The separated hydrogen may be pressurized to the pressure of the second reaction zone, and heated to or above the temperature of the second reaction zone. The separated hydrogen may be purified before recycling. In some embodiments, a gas-liquid separator may be used to separate the hydrogen from the effluent stream.

Similarly, water from the second reaction zone effluent may be recycled to one or more of a number of different locations within the process depending upon the specific embodiment employed. For example, in some embodiments at least a portion of the separated water may be recycled to combine with the whole cellulostic biomass containing input stream and/or saccharide process stream. In some embodiments, at least a portion of the separated water may be added to an optional pretreatment operation, and/or may be added to the second reaction zone. Further, in some embodiments the water may be purified before being recycled.

In some embodiments, the second reaction zone comprises a mixing zone upstream of the second reactor. In these embodiments, at least a portion of the separated hydrogen may be recycled directly to the reactor while at least a portion of the separated water is recycled to the mixing zone.

In some embodiments, the apparatuses described herein further comprise a product recovery zone where at least the polyols are separated from the second reaction zone effluent stream. Multiple product polyol streams may be produced by the product recovery zone. For instance, ethylene glycol may be separated into an ethylene glycol stream and propylene glycol may be separated into a propylene glycol stream. In some embodiments, one or more co-products are also separated from the second reaction zone effluent stream in the product recovery zone. For instance, co-products having a molecular weight lower than ethylene glycol, such as alcohols, may be separated into a low molecular weight co-product stream, co-products having a molecular weight higher than propylene glycol, such as glycerol, may be separated into a high molecular weight co-product stream, fuel gas may be separated into a fuel gas stream, and non-volatile residues may be separated into a non-volatile residue stream. In embodiments where at least one co-product stream is generated, a co-products stream may be recycled to the second reaction zone. In embodiments where the second reaction zone comprises a mixing zone upstream of a reactor, the recycled co-product stream may be directed to the reactor, the mixing zone, or both. Additionally, apparatuses described herein may further comprise a product purification zone, where one or more product polyol streams are purified to generate high purity polyol.

Depending upon the particulars of the saccharide-to-polyol catalyst system, the product recovery zone may also separate catalyst particles from the second reaction zone effluent stream. In some embodiments solid catalyst particles are removed from the effluent stream, either before or after recovery of products and/or co-products. Catalyst particles may be removed from the second reaction zone effluent stream using one or more techniques such as direct filtration, settling followed by filtration, hydrocyclone, fractionation, centrifugation, the use of flocculants, precipitation, extraction, evaporation, or combinations thereof. In some embodiments, catalyst particles are separated from the second reaction zone effluent stream after hydrogen is separated but before water is separated from the second reaction zone effluent stream. In some embodiments, separated catalyst particles are recycled to the second reaction zone. In some related embodiments, separated catalyst particles are reactivated before being recycled to the second reaction zone.

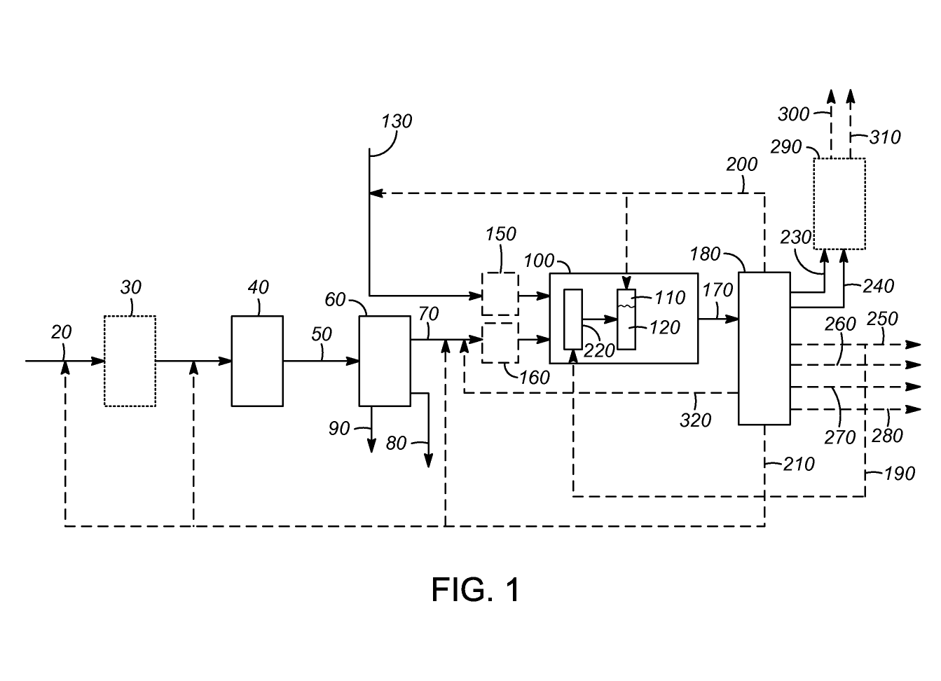

An exemplary method will now be described with reference to the flow scheme in FIG. 1. An input stream 20 comprising whole cellulostic biomass is optionally subjected to a pretreatment process comprising sizing, grinding, drying, or a combination thereof, in an optional pretreatment zone 30. The input stream 20 is then introduced into a first reaction zone 40, where the whole cellulostic biomass is treated to depolymerize at least a portion of the amount of lignin present. In some embodiments, the lignin depolymerization is conducted via hot water treatment, steam treatment, hydrolysis, pyrolysis, thermal treatment, chemical treatment, biological treatment, catalytic treatment, pressure treatment, and combinations thereof.

The first reaction zone 40 generates a first effluent 50 containing depolymerized lignin, saccharides (including e.g., cellulose), and metals and ash impurities. The first effluent 50 is directed to a first separation zone 60, where the first effluent is separated into at least two streams: a saccharide process stream 70 and a depolymerized lignin stream 80. In some embodiments, the first separation zone 60 also generates a waste stream 90 comprising ash and/or metal impurities from the whole cellulostic biomass. Separating the first effluent 50 into these respective streams may be conducted via any suitable separation technique.

The saccharide process stream 70 comprises one or more saccharides and is directed to a second reaction zone 100. The second reaction zone 100 is configured to receive the saccharide process stream 70 and combine the saccharide process stream 70 with one or more additional constituents (e.g., water and/or hydrogen) to generate a reaction mixture 120. The reaction mixture 120 is then contacted with a catalyst under conditions suitable for the catalytic conversion of saccharide to polyol. In some methods and as shown in FIG. 1, the saccharide process stream 70 is provided to the second reaction zone 100 separately from a hydrogen stream 130.

Depending on what, if any, pretreating steps are employed, water may already be present in the saccharide process stream 70. Further, in some embodiments, water may be added to the saccharide process stream 70, the hydrogen stream 130, or both, or water may be introduced to the second reaction zone 100 via an independent water stream (not shown in FIG. 1). In some embodiments, the hydrogen stream 130 may be heated in an optional hydrogen processing zone 150 prior to introduction to the second reaction zone 100. For instance, in some embodiments, hydrogen stream 130 may be heated to a temperature in excess of the reaction temperature of the second reaction zone 100. In some embodiments, the hydrogen stream 130 is heated to a temperature such that when the hydrogen stream 130 is introduced into a second reactor 110 (within the second reaction zone 100) and combined with the saccharide process stream 70, the temperature of the resulting reaction mixture 120 is at or above the necessary reaction temperature for a selected saccharide-to-polyol catalyst system. In particular, this allows for methods in which the temperature of the saccharide process stream 70 is controlled so as not to exceed the decomposition temperature of cellulose and/or so as not to exceed the charring temperature of cellulose, while still allowing a mixture of the saccharide process stream 70 and the hydrogen stream 130 to be at or above a necessary reaction temperature in the second reactor 110. Further, in some embodiments, the saccharide process stream 70 or the hydrogen stream 130 may be pressurized to a reaction pressure in an optional saccharide processing zone 160 or the optional hydrogen processing zone 150, respectively, before being introduced into the second reaction zone 100. In some embodiments, the saccharide process stream 70 and the hydrogen stream 130 are both pressurized to a reaction pressure.

In some methods, the second reactor 110 is a slurry reactor. In some related embodiments, a saccharide-to-polyol catalyst system is mixed with the saccharide process stream 70 and a sufficient amount of water to form a slurry prior to introduction into the second reactor 110. In some related embodiments, a slurry reactor may be operated at temperatures from about 100° C. to about 350° C. and the hydrogen pressure may be greater than about 150 psig. In some embodiments, the temperature in a slurry reactor may range from about 150° C. to about 350° C., such as about 200° C. to about 280° C.

In some related embodiments, the slurry reactor is operated with a water to saccharide weight ratio of about 1 to about 100, such as about 1 to about 20, such as about 1 to about 5. In some embodiments, the slurry reactor is operated with a catalyst to saccharide weight ratio of greater than about 0.005, such as greater than about 0.01, such as greater than about 0.1. In some embodiments, the slurry reactor is operated with a pH of less than about 10. In some embodiments, the slurry reactor is operated with a residence time of greater than 5 minutes. In some embodiments, the slurry reactor is operated such that any combination of these operating conditions are met.

In a specific exemplary embodiment, the slurry reactor is operated with a water to saccharide weight ratio of about 1 to about 20 and with a catalyst to saccharide weight ratio of greater than about 0.01. In yet another specific exemplary embodiment, the slurry reactor is operated with a water to saccharide weight ratio of about 1 to about 5 and with a catalyst to saccharide weight ratio of greater than about 0.1.

Within the second reaction zone 100 and at operating conditions, the reactants (i.e., saccharide, hydrogen, and water) proceed through catalytic conversion reactions to produce at least one polyol in a second reaction zone effluent 170. In some embodiments, the product polyol includes ethylene glycol, propylene glycol, or a combination thereof. In some embodiments, at least one co-product is also produced. Such co-products may include alcohols, organic acids, aldehydes, monosaccharides, polysaccharides, phenolic compounds, hydrocarbons, glycerol, depolymerized lignin, carbohydrates, and proteins. In some embodiments, a plurality of co-products may be produced. Some of the co-products may have commercial value and may be isolated and recovered in addition to the product polyols. Co-products may also be reaction intermediates.

Unreacted hydrogen, water, and saccharides may also be present in the second reaction zone effluent 170 along with co-products. In some embodiments, unreacted hydrogen, water, saccharides, or any combination thereof, are separated in a product recovery zone 180 and recycled back to the second reaction zone 100. In some embodiments, hydrogen is separated from the second reaction zone effluent 170 to generate a hydrogen recycle stream 200 before water is separated from the second reaction zone effluent 170 to generate a water recycle stream 210. The hydrogen recycle stream 200 may be recycled to one or more of a number of different locations within the process depending upon the specific embodiment employed. For example, the hydrogen recycle stream 200 may be recycled to the second reactor 110. As shown in FIG. 1, the hydrogen recycle stream 200 may be combined with fresh hydrogen or make-up hydrogen (e.g., as part of the hydrogen stream 130) before being introduced into second reactor 110. Alternatively or in addition (and not shown in FIG. 1) hydrogen recycle stream 200 may be introduced to second reactor 110 independently of fresh hydrogen or make-up hydrogen. Further, the hydrogen recycle stream 200 may be pressurized to the pressure of the second reaction zone 100, and heated to or above the temperature of the second reaction zone 100. The hydrogen recycle stream 200 may be purified before recycling. In some embodiments, a gas-liquid separator may be used to separate the hydrogen from the effluent stream.

Similarly, water from the second reaction zone effluent 170 may be separated via the product recovery zone 180 to form a water recycle stream 210 that may be recycled to one or more of a number of different locations within the process depending upon the specific embodiment employed. For example, in some embodiments at least a portion of a water recycle stream 210 is recycled to combine with the whole cellulostic biomass containing input stream 20 and/or saccharide process stream 70. In some embodiments, at least a portion of the water recycle stream 210 may be added to the input stream 20 prior to an optional pretreatment operation, and/or may be added to the second reaction zone 100. Further, in some embodiments the water recycle stream 210 may be purified via any suitable water purification system (not shown in FIG. 1) before being recycled.

In some embodiments, the second reaction zone 100 comprises a mixing zone 220 upstream of the second reactor 110. In these embodiments, at least a portion of the hydrogen recycle stream 200 may be recycled directly to the second reactor 110 while at least a portion of the water recycle stream 210 is recycled to the mixing zone 220.

In embodiments of the methods described herein, one or more product polyols are separated from the second reaction zone effluent 170 in the product recovery zone 180. Multiple product polyol streams may be generated in the product recovery zone 180. For instance, one or more polyol streams may include an ethylene glycol stream 230 and/or a propylene glycol stream 240.

In some embodiments, co-products may also be separated into one or more co-product streams, and optionally recycled back to the second reaction zone 100 as optional co-product recycle stream 190. In some embodiments, one or more co-product streams may include a low molecular weight co-product stream 250 comprising one or more co-products having a molecular weight lower than ethylene glycol, such as alcohols. In some embodiments, one or more co-product streams may include a high molecular weight co-product stream 260 comprising one or more co-products having a molecular weight higher than propylene glycol, such as glycerol. In some embodiments, one or more co-product streams may include a fuel gas stream 270. In some embodiments, one or more co-product streams may include a non-volatile residue stream 280. Additionally, apparatuses described herein may further comprise a product purification zone 290, where one or more product polyol streams are purified to generate one or more high purity polyol streams 300 and 310 by any suitable conventional purification technique.

Depending upon the particulars of the saccharide-to-polyol catalyst system, the product recovery zone 180 may also separate catalyst particles from the second reaction zone effluent 170. In some embodiments, catalyst particles 320 are separated from the second reaction zone effluent 170 either before or after recovery of products and/or co-products. Catalyst particles may be separated from the second reaction zone effluent 170 using one or more techniques such as direct filtration, settling followed by filtration, hydrocyclone, fractionation, centrifugation, the use of flocculants, precipitation, extraction, evaporation, or combinations thereof. In some embodiments, catalyst particles 320 are separated from the second reaction zone effluent 170 after generation of hydrogen recycle stream 200 but before of the water recycle stream 210. In some embodiments and as seen in FIG. 1, separated catalyst particles 320 are recycled to the second reaction zone 100. In some related embodiments, separated catalyst particles are reactivated before being recycled to the second reaction zone 100 (not shown in FIG. 1).

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method of catalytically generating a polyol from whole biomass, the method comprising the steps of:
depolymerizing at least a portion of lignin present in a stream comprising whole biomass;
generating an effluent comprising depolymerized lignin and a saccharide;
separating the effluent to generate a depolymerized lignin stream and a saccharide process stream, wherein the saccharide process stream comprises a saccharide and an amount of lignin that is reduced relative to an amount of lignin present the effluent; and
contacting the saccharide process stream with a saccharide-to-polyol catalyst system under conditions suitable for catalytic conversion of saccharide to polyol.

2. The method of claim 1, wherein the whole biomass comprises whole cellulostic biomass.

3. The method of claim 1, wherein the step of depolymerizing at least a portion of lignin present in a feedstock comprising whole biomass comprises treating the feedstock via hot water treatment, steam treatment, pyrolysis, thermal treatment, chemical treatment, biological treatment, catalytic treatment, pressure treatment, or combinations thereof.

4. The method of claim 3, wherein the chemical treatment comprises acid catalyzed hydrolysis or base catalyzed hydrolysis.

5. The method of claim 3, wherein the catalytic treatment comprises catalytic hydrolysis, catalytic hydrogenation, or both.

6. The method of claim 3, wherein the biological treatment comprises enzymatic hydrolysis.

7. The method of claim 1, wherein the step of separating the effluent further comprises generating a waste stream comprising ash and metal impurities from the whole biomass, wherein the saccharide process stream comprises amounts of ash and metal impurities that are reduced relative to amounts of ash and metal impurities present in the effluent.

8. The method of claim 1, wherein the saccharide-to-polyol catalyst system comprises two active metal components, wherein one of the active metal components is elemental platinum (Pt), elemental palladium (Pd), elemental ruthenium (Ru), or a combination thereof, and a second active metal component comprises molybdenum (Mo), tungsten (W), vanadium (V), nickel (Ni), cobalt (Co), iron (Fe), tantalum (Ta), niobium (Nb), titanium (Ti), chromium (Cr), zirconium (Zr), or any combination thereof, in an elemental state, a carbide compound, a nitride compound, a phosphide compound, or any combination thereof.

9. The method of claim 1, wherein the saccharide-to-polyol catalyst system is a compound catalyst system comprising an unsupported component comprising a compound selected from the group consisting of a tungsten (W) compound, a molybdenum (Mo) compound, and any combination thereof, and a supported component comprising an active metal component selected from the group consisting of platinum (Pt), elemental palladium (Pd), elemental ruthenium (Ru), rhodium (Rh), nickel (Ni), iridium (Ir), and combinations thereof on a solid catalyst support.

10. The method of claim 1, wherein the step of contacting the saccharide process stream with a saccharide-to-polyol catalyst system is conducted in an ebullating catalyst bed system, immobilized catalyst reaction system having catalyst channels, augured reaction system, fluidized bed reactor system, mechanically mixed reaction system, or slurry reactor system.

11. The method of claim 1, further comprising the step of pretreating the whole biomass via sizing, drying, grinding, or a combination thereof into solid particles of a size that may be flowed or moved through a continuous process using a liquid or gas flow, or mechanical processing, wherein the step of pretreating the whole biomass is conducted prior to the step of depolymerizing at least a portion of lignin in the whole biomass.

12. The method of claim 1, wherein the generated polyol comprises ethylene glycol, propylene glycol, or a combination thereof.

13. The method of claim 1, further comprising generating a second effluent, wherein the second effluent comprises a generated polyol and a co-product, wherein the generated polyol comprises ethylene glycol, propylene glycol, or a combination thereof, and the co-product is an alcohol, organic acid, aldehyde, monosaccharide, polysaccharide, phenolic compound, hydrocarbon, glycerol, depolymerized lignin, carbohydrate, or protein.

14. The method of claim 13, further comprising separating the second effluent to generate at least one polyol stream.

15. A method of catalytically generating a polyol from whole biomass, the method comprising the steps of:
depolymerizing at least a portion of lignin present in a stream comprising whole biomass;
generating a first effluent comprising depolymerized lignin and a saccharide;
separating the first effluent to generate a depolymerized lignin stream and a saccharide process stream, wherein the saccharide process stream comprises a saccharide and an amount of lignin that is reduced relative to an amount of lignin present the first effluent;
contacting the saccharide process stream with a saccharide-to-polyol catalyst system in a slurry reactor under conditions suitable for catalytic conversion of saccharide to polyol;
generating a second effluent comprising a polyol and saccharide-to-polyol catalyst;
separating saccharide-to-polyol catalyst from said second effluent; and
recycling the separated saccharide-to-polyol catalyst back to the slurry reactor.

16. The method of claim 15, wherein the whole biomass comprises whole cellulostic biomass.

17. The method of claim 15, wherein the step of depolymerizing at least a portion of lignin present in a feedstock comprising whole biomass comprises treating the feedstock via hot water treatment, steam treatment, pyrolysis, thermal treatment, chemical treatment, biological treatment, catalytic treatment, pressure treatment, or combinations thereof.

18. An apparatus for catalytic generation of a polyol from whole biomass, the apparatus comprising:
a first reaction zone configured to receive an input steam comprising whole biomass, subject the input stream to conditions suitable for depolymerization of lignin in the input stream, and generate a first effluent comprising depolymerized lignin and a saccharide;
a first separation zone in fluid communication with the first reaction zone; the first separation zone configured to receive the first effluent and separate the first effluent into a saccharide process stream and a depolymerized lignin stream, wherein the saccharide process stream comprises an amount of lignin that is reduced relative to an amount of lignin present in the first effluent; and
a second reaction zone in fluid communication with the first separation zone; the second reaction zone configured to receive the saccharide process stream, contact the saccharide process stream with a saccharide-to-polyol catalyst under conditions suitable for generating a polyol, and generate a second reaction zone effluent comprising a polyol.

19. The apparatus of claim 18, wherein the first separation zone is configured to separate the first effluent into a saccharide process stream, a depolymerized lignin stream, and a waste stream, wherein the saccharide process stream comprises an amount of lignin that is reduced relative to an amount of lignin present in the first effluent and amounts of ash and metal impurities that are reduced relative to amounts of ash and metal impurities present in the first effluent.

20. The apparatus of claim 18, wherein the second reaction zone comprises an ebullating catalyst bed system, immobilized catalyst reaction system having catalyst channels, augured reaction system, fluidized bed reactor system, mechanically mixed reaction system, or slurry reactor system.
